(19) United States
(12) Patent Application Publication
(10) Pub. No.: US 2021/0346544 A1
(43) Pub. Date: Nov. 11, 2021

(54) SYSTEMS, APPARATUS AND METHODS FOR MICROBICIDAL ULTRAVIOLET AND VIOLET BLUE LIGHT FOR MOTOR VEHICLES, HANDS AND STERILE INTRAOPERATIVE APPLICATION

(71) Applicant: Mark A. Anton, Newport Beach, CA (US)

(72) Inventor: Mark A. Anton, Newport Beach, CA (US)

(21) Appl. No.: 17/218,135

(22) Filed: Mar. 30, 2021

Related U.S. Application Data
(60) Provisional application No. 63/003,211, filed on Mar. 31, 2020.
(60) Provisional application No. 63/005,181, filed on Apr. 3, 2020.

(51) Int. Cl. A61L 2/10; A61L 2/24; B60Q 3/68

(52) U.S. Cl. CPC A61L 2/10; A61L 2/24; A61L 2202/11; A61L 2202/14; B60Q 3/68

(57) ABSTRACT

Provided are embodiments of system, apparatus and method for effectively sterilizing all types of vehicles as well as the hands. In some embodiments, the system, apparatus and method use UVC and/or far-UVC lights to sterilize the interior of the vehicle. In some embodiments, a device can sterilize the subungual spaces of the hands, using far-UVC lights.

### CROSS-REFERENCE TO RELATED APPLICATIONS

The present application claims priority to U.S. Provisional Patent Application No. 63/003,211, filed Mar. 31, 2020, and U.S. Provisional Patent Application No. 63/005,181, filed Apr. 3, 2020, the disclosures of both of which are hereby incorporated by reference in their entireties for all purposes.

### FIELD

The subject matter herein generally applies to microbicidal ultraviolet and violet blue light sterilization, and more particularly to microbicidal ultraviolet and violet blue light for all types of motor vehicles, hands and sterile intraoperative application.

### BACKGROUND

Motor vehicles are the mobile doorway to home, office and all stops in between. All microbes come along for the ride with all humans and their cargo to reach all possible destinations. The spread of microbial infections through these vehicular gateways needs to be eradicated. Motorized vehicles of land, water and air currently transport all humans and their cargo and there is no current, simple, cost effective way to kill the accompanying microbes (e.g., bacteria, viruses, fungi). Solutions, sprays, wipes can be used but they cannot be applied to all surfaces and crevices.

In addition, with the current pandemic, the spread of microbial infections via human hands also needs to be eradicated.

Accordingly, there is a need for a system, apparatus and method for effectively sterilizing all types of vehicles as well as the hands.

### SUMMARY

The present disclosure relates generally to a system, apparatus and method for effectively sterilizing all types of vehicles as well as the hands.

The microbicidal effects of ultraviolet (UV) light (far-UVC, UVC, UVB, UVA) and Violet-Blue (VB) light have been well established since 1934. One needs to give original credit to Downes and Blunt who in about 1877 discovered the antibacterial effects of sunlight. These same light wavelengths can also be used to eliminate the plethora of microflora growth on hands, especially the subungual space (under the fingernails). Another version of far-UVC light application to kill any and all microbes is via a sterile intraoperative far-UVC device, the light waves of which might be emitted by a diode or other forms of emitters.

UV light (far-UVC, UVC, UVB, UVA) and VB (violet-blue) light can be applied to practically all surfaces and the space in between (air). Various UV/VB wavelengths have different attributes (i.e., amount of exposure time needed to kill microbes) but all are unsafe for human exposure, except for far-UVC light. Far-UVC light wavelengths of the UV spectrum (207 nm-222 nm) are not carcinogenic or cataractogenic (harmful to the corneas), as they do not penetrate the upper most layers of the skin or eye.

In some embodiments, the present disclosure may apply UVC (or any UV/VB) wavelengths to motor vehicles to kill bacteria on the interior surfaces when people are not present and be applied to the air when people are in or out of the vehicle to kill any airborne microbes. For example, these lights may be automatically turned on when the car is not in use so that when the car is again used, the microbial load is significantly decreased.

In some embodiments, as the far-UVC light also kills microbes without harm to humans, it may be turned on when the vehicle is in use, thus helping to kill the microbes that are introduced into the vehicle via the skin, clothes and cargo of the occupants.

In some embodiments, the present disclosure may include a process for sanitizing or sterilizing a vehicle. The process may include determining whether there is any occupant inside the vehicle. Upon determining that there is no occupant inside the vehicle, applying UVC light to one or more interior surface of the vehicle. Otherwise, upon determining that there is an occupant inside the vehicle, applying far-UVC light to an interior of the vehicle.

In some embodiments, a device of the present disclosure may allow far-UVC to be used for the hands and fingernails, as it would be harmless to those tissues.

This summary and the following detailed description are merely exemplary, illustrative, and explanatory, and are not intended to limit, but to provide further explanation of the invention as claimed. Additional features and advantages of the invention will be set forth in the descriptions that follow, and in part will be apparent from the description, or may be learned by practice of the invention. The objectives and other advantages of the invention will be realized and attained by the structure particularly pointed out in the written description, claims and the appended drawings.

### BRIEF DESCRIPTION OF THE DRAWINGS

The present invention may be better understood by referring to the following FIGURES. The components in the figures are not necessarily to scale. Emphasis instead being placed upon illustrating the principles of the disclosure. In the FIGURES, reference numerals designate corresponding parts throughout the different views.

FIG. 1 shows an example diagram of a process for sterilizing a vehicle, according to some embodiments of the present disclosure.

### DETAILED DESCRIPTIONS

The following disclosure describes various embodiments of the present invention and method of use in at least one of its preferred, best mode embodiments, which is further defined in detail in the following description. Those having ordinary skill in the art may be able to make alterations and modifications to what is described herein without departing from its spirit and scope. While this invention is susceptible to different embodiments in different forms, there is shown in the drawings and will herein be described in detail a preferred embodiment of the invention with the understanding that the present disclosure is to be considered as an exemplification of the principles of the invention and is not intended to limit the broad aspect of the invention to the embodiment illustrated. All features, elements, components, functions, and steps described with respect to any embodiment provided herein are intended to be freely combinable and substitutable with those from any other embodiment unless otherwise stated. Therefore, it should be understood that what is illustrated is set forth only for the purposes of example and should not be taken as a limitation on the scope of the present invention.

As used herein and in the appended claims, the singular forms “a,” “an,” and “the” include plural referents unless the context clearly dictates otherwise.

In general, terms such as “coupled to,” and “configured for coupling to,” and “secure to,” and “configured for securing to” and “in communication with” (for example, a first component is “coupled to” or “is configured for coupling to” or is “configured for securing to” or is “in communication with” a second component) are used herein to indicate a structural, functional, mechanical, electrical, signal, optical, magnetic, electromagnetic, ionic or fluidic relationship between two or more components or elements. As such, the fact that one component is said to be in communication with a second component is not intended to exclude the possibility that additional components may be present between, and/or operatively associated or engaged with, the first and second components.

In the following description and in the FIGURES, like elements are identified with like reference numerals. The use of “e.g.,” “etc.,” and “or” indicates non-exclusive alternatives without limitation, unless otherwise noted. The use of “including” or “includes” means “including, but not limited to,” or “includes, but not limited to,” unless otherwise noted.

As used herein, the term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “comprising” can refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

Provided herein are example embodiments of system, apparatus and method where UVC (or any UV/VB) wavelengths can be applied to motor vehicles to kill bacteria on the interior surfaces.

In some embodiments, the present disclosure may apply UVC (or any UV/VB) wavelengths to motor vehicles to kill bacteria on the interior surfaces when people are not present and be applied to the air, for example running through the vents of a car, when people are in or out of the vehicle to kill any airborne microbes. For example, these lights may be automatically turned on when the vehicle is not in use so that when the vehicle is again used, the microbial load is significantly decreased.

In some embodiments, the present disclosure may include system, apparatus and method for applying UV/VB light to all interior surfaces, including air flow of any vehicle whether it is on land, rail, water or air. For example, one or more interior UV/VB lights may be installed above or on top of any surface, embedded in any surface, and/or hidden underneath overhangs. They may also be installed within the air flow (e.g., A/C or air cooler) ducts.

In some embodiments, far-UVC light may be used in vehicles as it can be used any time of day or night with or without occupants being present. The other UV/VB light wavelengths may be used when there is no possibility of people in or around the vehicle, but it is difficult to control those variables in a vehicle as opposed to, for example, an enclosed building. If and when these wavelengths (non-far-UVC), are used, they may be oriented in a way to minimize any outside direct line of vision, thus protecting a person's eyes that may be near the vehicle.

In some embodiments, external UV/VB light may be situated above/around the door handles (with or without an overhang to protect one's eyes) so as to kill microbes that land on these surfaces. With automatic door handles, for example in a Tesla car, the exposed surface of the handle may be retracted when not in use to allow a light imbedded inside the enclosing edge to shine on the surface for a programmed amount of time. When the UV/VB light exposure is sufficient, the light turns off and the handle moves outward to the starting flush position. Outside air ducts may also have UV/VB lights to kill microbes entering from this approach.

In some embodiments, certain interior vehicle surfaces may need to be optimized to minimize any color, texture, etc., changes that UV/VB lights can create.

The microbial load to which a human is exposed is directly related to the severity of the disease they might contract. As such, decreasing or eliminating the microbial load of a vehicular, for example the vehicle doorway, will significantly decrease the transfer and spread of infections at all times. When used routinely, UVC light (electromagnetic spectrum) will also kill the normal microbial growth that occurs in these areas. The use of UVC light to kill microbes is superior to biological killing of these microbes as mutations in the genetic material (new strains) would not change its effectiveness. In some embodiments, the present disclosure may include using UVC light especially in the A/C coils and air flow.

Cargo in a truck bed and trunk of a vehicle can also be treated with UV/VB light thus also killing transfer of microbes with packages.

In some embodiments, as the far-UVC light also kills microbes without harm to humans, it can be turned on when the vehicle is in use, thus helping to kill the microbes that are introduced into the vehicle via the skin, clothes, and cargo of the occupants. The microbicidal effects of far-UVC can be effectively applied within the time period of usual car rides. Far-UVC is far superior to UVC (and UVA/UVB) for disinfection as it is more efficient at lower dose rates, kills pathogens in seconds rather than minutes, disrupts nucleic acids (DNA/RNA) and their bonds much better so as to prevent them from replicating/functioning properly and is safe to use around humans/animals (e.g., it does not penetrate skin or eyes).

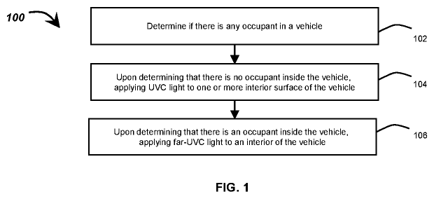

FIG. 1 shows an example diagram of a process 100 for sterilizing a vehicle. In some implementations, the process may include determining whether there is any occupant inside the vehicle. Upon determining that there is no occupant inside the vehicle, applying UVC light to one or more interior surface of the vehicle. On the other hand, upon determining that there is an occupant inside the vehicle, applying far-UVC light to an interior of the vehicle.

Provided herein are also example embodiments of a device that may allow far-UVC to be used for the hands and fingernails.

Far-UVC can also be used for the hands and fingernails, as it would be harmless to those tissues. The subungual space has been well-documented as harboring more bacteria and the growth of other microbes than the skin of the hands. This space can be debulked with proper nail brushing along with soap and microbicidal agents, but it is difficult to kill all the microbes. A device of the present disclosure that allows the placement/insertion of just the fingertips (or the entire hands) and that has an integrated far-UVC emitter, would kill the preponderance of microbes, depending on the length of use. This device can be used in all areas that humans traverse from homes, motor vehicles, businesses and everywhere in between. It may become standard medical and surgical equipment in all medical and surgical facilities, especially for those scrubbing into surgery.

The device of the present disclosure may also become a standard device to be used in all medical and surgical facilities to better reduce all microflora of the hands and subungual space, thus greatly reducing the transfer of microbes through touch.

In some embodiments, UVC/Far-UVC lights may be used intraoperatively with a sterile wand for areas clinically or sub-clinically infected or contaminated (e.g., capsular contracture of a silicone implant). The device may provide an ideal dose that may kill various microbes but may be harmless or minimally harmless to exposed tissues.

In some embodiments, the present disclosure may include sterile UVC/Far-UVC emitting diodes embedded in a catheter-like device which, for example, may be inserted percutaneously to reduce or eliminate bodily infections (e.g., sinusitis, peri-implant infection, etc.).

In some embodiments, the present disclosure may include applying UVC/Far-UVC light in reducing wound infections in surgical sites, for example, by routinely applying these wavelengths to the wounds before closure. Approximately 200,000 to 300,000 patients suffer surgical wound infections in the U.S. each year, accounting for $3 billion to $10 billion in health-care expenditures. UVC/Far-UVC light might be especially helpful in any traumatic wound developed outdoors or outside of a medical setting.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed is:

1. A method for sterilizing a vehicle, comprising:
determining whether there is any occupant inside the vehicle;
upon determining that there is no occupant inside the vehicle, applying UVC light to one or more interior surface of the vehicle; and
upon determining that there is an occupant inside the vehicle, applying far-UVC light to an interior of the vehicle.

2. The method of claim 1, wherein UVC lights are installed above the one or more interior surface of the vehicle.

3. The method of claim 1, wherein UVC lights are embedded in the one or more interior surface of the vehicle.

4. The method of claim 1, wherein UVC lights are hidden underneath one or more overhangs in the interior of the vehicle.

5. The method of claim 1, wherein UVC lights are further installed with one or more door handle of the vehicle.

6. The method of claim 1, wherein UVC lights are automatically turned on when the vehicle is not in use.

7. The method of claim 1, wherein far-UVC lights are installed in an A/C coil of the vehicle.

8. The method of claim 1, wherein far-UVC lights are installed in one or more air flow duct of the vehicle.

9. The method of claim 1, wherein UVC lights are further installed with a trunk of the vehicle.

10. The method of claim 1, wherein UVC lights are further installed with a truck bed of the vehicle.

11. A device for sterilizing a hand, comprising:
a wand, wherein the wand emits microbicidal lights.

12. The device of claim 11, wherein the wand emits UVC lights.

13. The device of claim 11, wherein the wand emits far-UVC lights.

14. A device for sterilizing a hand, comprising:
a catheter-like device, wherein the catheter-like device includes embedded ultraviolet light diodes.

15. The device of claim 14, wherein the embedded ultraviolet light diodes emit UVC lights.

16. The device of claim 14, wherein the embedded ultraviolet light diodes emit far-UVC lights.
